(19) United States
(12) Patent Application Publication
(10) Pub. No.: US 2025/0139134 A1
(43) Pub. Date: May 1, 2025

(54) METHOD AND APPARATUS FOR MONITORING CARBON SEQUESTERED FROM GREEHNOUSE GAS EMISSIONS

(71) Applicant: ARC CARBON CORPORATION, Winchester, VA (US)

(72) Inventors: John S EBERHARDT, III, Winchester, VA (US); Jason S EBERHARDT, Stanhope, NJ (US); Benjamin S FELDMAN, Belchertown, MA (US)

(73) Assignee: Benjamin S. FELDMAN, Belchertown, MA (US)

(21) Appl. No.: 18/681,591

(22) PCT Filed: Aug. 4, 2022
(86) PCT No.: PCT/US2022/039480

§ 371 Date: Feb. 6, 2024

Related U.S. Application Data
(60) Provisional application No. 63/233,417, filed on Aug. 16, 2021.

(51) Int. Cl. G06F 16/29; G06F 17/40; G06Q 40/04; G16C 20/20

(52) U.S. Cl. CPC G06F 16/29; G06F 17/40; G16C 20/20; G06Q 40/04

(57) ABSTRACT

A monitoring system tracks greenhouse gas capture and emission from plural source locations. The monitoring system comprises multiple computational systems, which use one or more structured transformation models (STMs) to create an ongoing, self-referencing, self-updating, and continuous state monitoring and recording system for tracking the entire carbon sequestration lifecycle including the re-release of sequestered carbon. The monitoring system receives baseline and periodically updated, captured, and emitted carbon sensor data, including location, physical characteristics of the carbon and ownership from registered asset owners. The system analyzes plural ADS and other categorized data to derive any temporal changes in ownership, quantity and/or physical properties of sequestered carbon data in a continuous feedback loop. A digital ledger stores carbon data in codified data structures, in compliance with tradeable carbon credit registration regulations.

### PRIORITY CLAIM

This application claims the benefit of priority of U.S. Provisional Application No. 63/233,417, filed Aug. 16, 2021, and entitled “Method and Apparatus for Monitoring Carbon Sequestered from Greenhouse Gas Emissions” which is incorporated by reference herein.

### STATEMENT REGARDING FEDERALLY SPONSORED RESEARCH

Development of the subject matter of this application has not been supported by the United States Department of Agriculture (“USDA”). Accordingly, the United States Government has no rights in this application. The assignee of this application has submitted Proposal No. USDA-NRCS-COMM-22-NOF00001139 under the USDA's Partnerships for Climate-Smart Commodities program to support further development of its technology, but as of the filing date of this application the USDA has not awarded funding.

### TECHNICAL FIELD

The disclosure relates to instrumentation and systems for monitoring reductions in greenhouse gas emissions by measuring and tracking temporal changes in location and quantities of carbon sequestered from atmospheric methane and carbon dioxide. Sequestered carbon data are imputed to a dynamically updated database, which maintains a digital ledger of carbon deposits and withdrawals. More particularly, in some embodiments, the disclosure enables smaller farm, ranch and forestry operations to self-monitor the quantities of carbon sequestered by plants and trees, both above and below ground, on their managed properties and report same to an easily accessible registry.

### BACKGROUND

There is current worldwide interest in reducing the rate of accumulation of greenhouse gas emissions into the atmosphere. Exemplary greenhouse emissions of interest include, but are not limited to methane and carbon dioxide, both of which contain carbon. One proposed type of technical solution to reduce concentrations of greenhouse gasses in the atmosphere is to sequester its carbon content prior to atmospheric emission. Carbon content is separated from the emissions and converted into other compositions of matter, such as building materials, algae, or biofuel. Another proposed type of technical solution is to extract carbon from carbon dioxide in the atmosphere, through plant photosynthesis, including forestry and sequester the carbon in the plants.

Monitoring, quantifying. tracking and recording sequestered carbon data accurately has been technically challenging, unreliable, and expensive. For example, many of the aforementioned carbon sequestration methods also directly and indirectly release carbon back into the atmosphere, e.g., consumed fuel, fertilizer, and electricity. Carbon capture in agricultural plants is temporal once the plants are harvested for food. Similarly, carbon captured in trees is temporal if they burn in a forest fire. A portion of carbon captured in harvested trees remains captured in lumber, but if a building incorporating the lumber burns down, carbon is re-released to the atmosphere.

Carbon sequestration, by whatever technical solution, incurs economic costs that must be recouped, in order to achieve economically viable solutions to reduction of greenhouse gas emissions. One proposed economic model to pay for greenhouse gas emission reduction, adopted by various governmental bodies, is to set maximum carbon emission quotas and/or tax the carbon content, e.g., per ton of carbon dioxide emitted. Emitters are incentivized to reduce carbon emissions in order to reduce their carbon tax payments and/or avoid penalties for exceeding emission quotas. In parallel, other proposed economic models incentivize adoption of carbon emission reduction, such as through use of non-fossil fuel energy, and/or carbon sequestration technologies. Those who reduce greenhouse gas emissions receive carbon credits, for example per ton of carbon dioxide that was not emitted into the atmosphere. Carbon credits are bought and sold on financial exchanges, but there is no central registry, equivalent to an accounting ledger or a digital wallet, where quantities, physical characteristics, locations, and ownership of the actual sequestered carbon-containing materials are tracked and recorded. Existing central registries do not accurately record physical characteristics of stored carbon, but rather they estimate captured carbon quantities based on the type and method of sequestration.

With respect to non-fossil fuel, alternative electrical-energy production, such as by solar, wind or nuclear power sources, it is relatively easy to measure and monitor the kilowatt hours of energy produced. It is also possible to estimate carbon emission reduction compared with the equivalent kilowatts produced in a traditional fossil-fueled power plant or vehicle. In fact, alternative energy carbon credits are bought and sold in financial markets. From an economic and financial perspective, produced alternative energy has a primary discernable value based on market price of a kilowatt hour of electricity and a separable, secondary discernable value based on market price for the carbon credit. Each are capable of being separately traded in financial markets. The challenge has been finding ways to identify and legally record existence and ownership sequestered carbon accurately, as is done for real estate, motor vehicles, company stock, bonds, and currency.

An example of a carbon-sequestering, tangible good with a secondary discernible and non-separable characteristic of sequestered carbon value is timber. While fast growth pine can be harvested for flooring and other building materials, a pine tree's natural ability to ingest and sequester carbon dioxide through photosynthesis allows the harvested tree and its resulting material to become a long-term storage medium for a greenhouse gas, regardless of when the material is transmuted from tree to lumber. While the tree may be harvested and apportioned as varied sizes and cuts of lumber, the physical storage of the greenhouse gas CO₂ is not, at present, separable and apportionable separately from the initial tree. While businesses have developed for the sale of forest-based CO₂ storage, they are unable to remove the trees from the forest, as once the tree is cut, its potential use as a storage medium is terminated and the value of the sequestered carbon cannot be separated and apportioned apart from the physical tree. While the timber itself is apportionable and the volume of stored CO₂ within the timber is also apportionable pari passu with the portions of timber, currently they are intrinsically tied together. There is no central registry that is able to track the sequestered carbon content of lumber or other tree products as they are transformed into other materials and transported to other locations.

Turning to quantification of sequestered carbon and its valuation for reduction of greenhouse gas emission in other agricultural industries, presently agricultural carbon credits are registered and validated against standards and protocols which are based on assumptive principles around large-scale projects, rather than scientifically measured volumes of carbon in related biomass (soil, foliage, timber, etc.). Existing carbon credit registries and validation organizations burden the agricultural industry with reporting, process, and ongoing business requirements dissuading or outright ignoring small agricultural businesses with less than 150 acres.

With nearly 90% of US farms generating less than US $350,000 per year and operating approximately 49% of farmland—while generating less than 21% of total production—the need for a minimal administrative burden and overhead, sequestered carbon credit registration, validation, and commodity management system is beneficial for both agricultural operators and the environment.

### SUMMARY

A universally accessible, easy to use monitoring system tracks greenhouse gas capture and emission from plural source locations. The system facilitates easy, universal, remote input of sequestered and emitted carbon data by agricultural growers, green industry builders, industrial manufacturing, and other businesses. The computer server-based monitoring system receives captured and emitted carbon sensor data, including location, physical characteristics of the carbon and ownership (asset data set or “ADS”) from registered remote users (registered asset owners or “RAO”) via an application programming interface of a data acquisition system and stores same in a registration database.

The monitoring system, executed on the server, is a model-based state engine, comprising multiple computational systems, which include a continuous monitoring engine, a registration database, a transactional digital ledger, and a carbon continuous settlement engine (also referred to as a digital wallet). Each computational system supports unique mechanisms, which use one or more structured transformation models (STMs) specified in computer object code, representing both Gaussian and logical transforms that interoperate with other similar mechanisms and computational systems to create an ongoing, self-referencing, self-updating, and continuous state monitoring and recording system for tracking the entire carbon sequestration lifecycle including the re-release of sequestered carbon.

In the monitoring system, the continuous monitoring engine executed on the server, in communication with the registration database, defines and receives from the RAO, or its designated third parties, initial baseline data needed to quantify the physical characteristics and quantity of sequestered carbon at the registered location. Thereafter, the continuous monitoring engine periodically communicates with the RAO, or its designated third parties, to receive and collect new data for an existing ADS, including any changes in any one or more of: quantity of sequestered carbon attributable to physical processes of additional sequestration from and/or release of either previously sequestered or new carbon sources into the environment, and/or transformation of physical properties, and/or transport into or away from the registered sequestration location and/or ownership.

Conceptually, the continuous monitoring engine maintains and archives a temporal ledger of changes in the quantity of sequestered carbon at a registered location. It debits the carbon asset ledger for any increases in carbon emissions at the site or for transportation of sequestered carbon away from the registered location. It credits the carbon asset ledger for any increases in the amount of carbon sequestered at the site, e.g., from growing plants or embedding carbon in building materials. When carbon is transported to a different location, it is tracked at the new location and any future locations where it is transported during its entire lifecycle. In the example of non-carbon power generation, in some embodiments the continuous monitoring engine tracks the location of equivalent carbon that would have otherwise been generated by a fossil-fuel power plant.

The continuous monitoring engine conforms the newly received data, from the RAO or its designated third parties, to its internal data interchange format, and updates the ADS. When required for the ADS, the continuous monitoring engine assigns one or more stock keeping units (SKU) to sub-units of the sequestered carbon to monitor material transformation and/or transport thereof in stream of commerce (for example, assigning an SKU to a new pallet of lumber obtained from a harvested tree and deleting the previously assigned SKU for that tree) and communicates the SKU data to the RAO, via its user communication device, and/or an ERP system of the RAO.

The continuous monitoring engine, in communication and conjunction with the digital ledger, generates sequestered carbon asset units (SCAUs), linked to each ADS, on behalf of the RAO. An SCAU is the tradable and transferrable unit of carbon, functionally equivalent to a unit of currency or a share of stock. Each generated SCAU includes origination location and current location of sequestered carbon. The continuous monitoring engine aggregates and analyzes plural ADS, SKU and SCAU data to derive any temporal changes in ownership, quantity and/or physical properties of sequestered carbon attributable to: additional sequestration from, and/or release of previously sequestered carbon, and/or introduction of new carbon sources into the environment, transformation of physical properties of the sequestered carbon, and/or transport of sequestered carbon into or away from the registered sequestration location. The continuous monitoring engine updates each ADS, SKU and SCAU data in a continuous feedback loop.

A digital ledger of the monitoring system, in communication with the continuous monitoring engine, stores the SCAU data in codified data structures that are in compliance with governmental requirements for the generation of tradeable carbon credits. The digital ledger updates, in a continuous feedback loop, the SCAU data, and the codified data structures for compliance with changes in the governmental requirements. The SCAU codified data structures of the digital ledger are a reliable, accurate repository and archive of geographic and temporal changes in greenhouse gas emissions and reductions based on monitoring of net quantities of sequestered carbon. The SCAU data are accessed by a carbon continuous settlement engine that is in communication with the digital ledger. The carbon settlement engine is accessible by governmental regulators who monitor compliance with greenhouse gas emissions laws, scientists, and financial intermediaries who are responsible for trading carbon credits.

Exemplary embodiments of the disclosure feature a sequestered carbon monitoring and data archiving system, comprising a server including one or more processors coupled to one or more computer-readable, non-transitory storage media. The media include therein instructions to operate, when executed by the one or more processors, to cause the server to create and store in the non-transitory storage media in an internal data interchange format, a registration database incorporating plural asset data sets (ADS), each including: a registered asset owner and/or its designated third party (RAO) identifier, a geographic location identifier, and carbon content specifications required to record physical characteristics and quantity of sequestered carbon. The registration database also incorporates RAO access credentials for accessing the server.

The monitoring system generates a new ADS in the registration database, in response to a user request to become an RAO. The user request is communicated to a portal of the server, via an application programming interface (API) or enterprise resource planning (ERP) software operating on a communication device of the user. The server communicates RAO access credentials to the user and now recognizes the user as an RAO.

The server operates a continuous monitoring engine (CME), created, and stored in the non-transitory storage media, in communication with the registration database. The CME processes each ADS as follows.

Upon initial registration of a new ADS, the CME defines initial baseline data needed to quantify the physical characteristics and quantity of sequestered carbon at the registered location.

The CME communicates a request for the defined initial baseline data to and receives them from the RAO, via its user communication device, and conforms the received initial baseline data to its internal data interchange format.

The CME periodically communicates with the RAO or its designated third parties, via its user communication device, to receive and collect new data for an existing ADS, including any changes in any one or more of: quantity of sequestered carbon attributable to physical processes of additional sequestration from and/or release of either previously sequestered or new carbon sources into the environment, and/or transformation of physical properties, and/or transport into or away from the registered sequestration location and/or ownership thereof, conforms the newly received data to its internal data interchange format, and updates the ADS.

The CME communicates with the RAO or its designated third parties, via its user communication device, requesting new data to update the existing ADS to conform with modification of ADS specifications or requirements used in operation of the CME, receives the new data from the RAO, conforms the newly received data to its internal data interchange format, and updates the ADS.

The CME, when required for the ADS, assigns one or more stock keeping units (SKU) to sub-units of the sequestered carbon to monitor material transformation and/or transport thereof in stream of commerce and communicates the SKU data to the RAO, via its user communication device.

The CME, in conjunction with the digital ledger, generates carbon sequestration asset units (SCAUs), linked to each ADS. Each SCAU includes quantity, quality, characteristics, origination location and current location of sequestered carbon.

The CME communicates with external data services, including both sensors and external computer accounting systems, associated with specific ADS in the registration database, to collect component input data in native format.

The CME transforms data throughout the continuous monitoring process using a model-based interpreter that uses one or more structured transformation models (STMs) specified in object code representing both Gaussian and logical transforms;

The CME stores STMs as self-referencing data objects that expose input-output metadata such that the model-based interpreter can automatically load the relevant STMs for a given set of data for a combination of ADS and SCAU, such that the interpreter will read the stored data objects and perform Gaussian and logical transforms at runtime.

The CME specifies STMs that perform Gaussian and logical transformations on the input data to support physical property calculations, legal compliance tests, and accounting allocations;

The CME transforms ADS component data to normalized elements for aggregation and analysis using a model-based interpreter that specifies normalizations specific to data input source and data elements.

The CME aggregates and analyzes plural ADS, SKU and SCAU data to derive any temporal changes in ownership, quantity and/or physical properties of sequestered carbon attributable to: additional sequestration from, and/or release of previously sequestered carbon, and/or introduction of new carbon sources into the environment, transformation of physical properties of the sequestered carbon, and/or transport of sequestered carbon into or away from the registered sequestration location using applied STMs in the model-based interpreter. The CME updates each ADS, SKU and SCAU data in a continuous feedback loop; and

The server creates a digital ledger, in the non-transitory storage media, which is in communication with the continuous monitoring engine. The digital ledger stores the SCAU data in codified data structures that are in compliance with governmental requirements for the generation of tradeable carbon credits, and it updates, in a continuous feedback loop, the SCAU data, and the codified data structures for compliance with changes in the governmental requirements.

Other exemplary embodiments of the disclosure feature a method for monitoring and generating asset data sets (ADS) concerning properties and characteristics of sequestered carbon and archiving same in a database. In exemplary embodiments, the method is practiced in a server including one or more processors coupled to one or more computer-readable, non-transitory storage media. The media include therein instructions to operate when executed by the one or more processors. When executing the instructions, the server creates and stores in one of its non-transitory storage media, in an internal data interchange format, a registration database incorporating plural asset data sets (ADS). Each ADS includes: a registered asset owner and/or its designated third party (RAO) identifier, a geographic location identifier, and carbon content specifications required to record physical characteristics and quantity of sequestered carbon. The server also incorporates RAO access credentials for accessing the server in the registration database.

The instructions specify to the server Gaussian and/or logical transformation models in object code stored as data in one of its non-transitory storage media, the models representing normalization procedures for input data; logical checks for legal compliance; and allocations for accounting purposes; which models will be interpreted by executable code at runtime.

When executing the instructions, the server generates a new ADS in the registration database, in response to a user request to become an RAO, with the user request communicated to a portal of the server, via an application programming interface (API) or enterprise resource planning ERP software operating on a communication device of the user. The server communicates RAO access credentials to the user and now recognizes the user as an RAO;

When executing the instructions, the server operates a continuous monitoring engine, created and stored in one of its non-transitory storage media, in communication with the registration database, which for each ADS:
upon initial registration of a new ADS, defines initial baseline data needed to quantify the physical characteristics and quantity of sequestered carbon at the registered location;
communicates a request for the defined initial baseline data to and receives them from the RAO or its designated third parties, via its user communication device, and conforms the received initial baseline data to its internal data interchange format, and stores same in one of its non-transitory storage media;
periodically communicates with the RAO or its designated third parties, via its user communication device, to receive and collect new data for an existing ADS, including any changes in any one or more of: quantity of sequestered carbon attributable to physical processes of additional sequestration from and/or release of either previously sequestered or new carbon sources into the environment, and/or transformation of physical properties, and/or transport into or away from the registered sequestration location and/or ownership thereof, conforms the newly received data to its internal data interchange format, and updates the ADS in one of its non-transitory storage media;
communicates with the RAO or its designated third parties, via its user communication device, requesting new data to update the existing ADS to conform with modification of ADS specifications or requirements used in operation of the continuous monitoring engine, receives the new data from the RAO, conforms the newly received data to its internal data interchange format, updates the ADS, and stores same in one of its non-transitory storage media.

When executing the instructions, the server, when required for the ADS, assigns one or more stock keeping units (SKU) to sub-units of the sequestered carbon to monitor material transformation and/or transport thereof in stream of commerce and communicates the SKU data to the RAO, via its user communication device, and stores same in one of its non-transitory storage media.

When executing the instructions, the server generates carbon sequestration asset units (SCAUs), linked to each ADS, each SCAU including quantity, quality, characteristics, origination location and current location of sequestered carbon and stores same in one of its non-transitory storage media.

When executing the instructions, the server communicates with external data services, including both sensors and external computer accounting systems, associated with specific ADS in the registration database, to collect component input data in native format, and stores same in one of its non-transitory storage media.

When executing the instructions, the server transforms data throughout the continuous monitoring process using a model-based interpreter that uses one or more structured transformation models (STMs) specified in object code representing both Gaussian and logical transforms, and stores same in one of its non-transitory storage media. The server also stores STMs as self-referencing data objects that expose input-output metadata such that the model-based interpreter can automatically load the relevant STMs for a given set of data for a combination of ADS and SCAU such that the interpreter will read the stored data objects and perform Gaussian and logical transforms at runtime, and stores same in one of its non-transitory storage media.

When executing the instructions, the server specifies STMs that perform Gaussian and logical transformations on the input data to support physical property calculations, legal compliance tests, and accounting allocations, and stores same in one of its non-transitory storage media.

When executing the instructions, the server transforms ADS component data to normalized elements for aggregation and analysis using a model-based interpreter that specifies normalizations specific to data input source and data elements, and stores same in one of its non-transitory storage media; and aggregates and analyzes plural ADS, SKU and SCAU data to derive any temporal changes in ownership, quantity and/or physical properties of sequestered carbon attributable to: additional sequestration from, and/or release of previously sequestered carbon, and/or introduction of new carbon sources into the environment, transformation of physical properties of the sequestered carbon, and/or transport of sequestered carbon into or away from the registered sequestration location using applied STMs in the model-based interpreter, storing the aggregation and analysis data in in one of its non-transitory storage media.

When executing the instructions, the server updates each ADS, SKU and SCAU data in a continuous feedback loop and stores same in one of its non-transitory storage media; and

When executing the instructions, the server creates a digital ledger, in one of its non-transitory storage media, which is in communication with the continuous monitoring engine, and stores in the digital ledger the SCAU data in codified data structures that are in compliance with governmental requirements for the generation of tradeable carbon credit. The server updates, in the digital ledger in a continuous feedback loop in conjunction with the continuous monitoring engine, the SCAU data, and the codified data structures for compliance with changes in the governmental requirements.

The respective features of the exemplary embodiments that are disclosed herein may be applied jointly or severally in any combination or sub-combination.

### BRIEF DESCRIPTION OF DRAWINGS

The exemplary embodiments of the disclosure are further described in the following detailed description in conjunction with the accompanying drawings, in which:

FIG. 1 is a lifecycle flow chart of the greenhouse gas emission and sequestration monitoring process practiced by the monitoring system of the present disclosure;

FIG. 2 is a block diagram of system architecture of the greenhouse gas emission and sequestration monitoring system of the present disclosure, as implemented in a computer server;

FIG. 3 is a process block diagram of the monitoring system of FIG. 2, showing interaction of the measurement standardization model implemented in the registration database for recording, obtaining and updating carbon asset data streams (ADS), the legal conformance model implemented within the model repository for registering legally recognized carbon assets, the continuous monitoring engine for mapping and updating stock keeping units (SKU's) and carbon sequestration asset units (SCAUs), linked to each ADS, and the digital ledger for recording SCAUs;

FIG. 4 is a block process diagram of the legal conformance model, implemented in the model repository of FIG. 2;

FIG. 5 is a process block diagram of the measurement standardization model, implemented in the registration database of the monitoring system of FIG. 2, for inventorying and recording baseline carbon data for registered geographic location parcels;

FIG. 6 is process block diagram of the monitoring system of FIG. 2, showing how the continuous monitoring engine models the lifecycle carbon state in plants and soil grown on an exemplary registered farm parcel; and

FIG. 7 is a process block diagram of the monitoring system of FIG. 2, showing the continuous monitoring engine continuously tracks and updates SKUs and SCAUs in an embodiment for monitoring the life cycle of carbon sequestered in forest trees, and subsequent transformation/transportation of timber to lumber to building construction site in a chain of commerce.

To facilitate understanding, identical reference numerals have been used, where possible, to designate identical elements that are common to the figures. The figures are not drawn to scale.

### DESCRIPTION OF EMBODIMENTS

Exemplary embodiments of the disclosure are utilized in a scientifically measurable and repeatable monitoring system for the accurate validation, registration, long term monitoring, and management, of sequestered carbon dioxide, through agricultural and other storage mediums. This carbon monitoring system architecture facilitates registration and tracking of sequestered carbon with the asset transfer and accounting functions used in traditional commodities, so that the positive environmental byproduct of agricultural photosynthesis, and other atmospheric carbon-removal solutions, and their inherent carbon sequestration, can be quantified, measurably commoditized, and effectively applied as a direct environmental offset against CO₂ emissions, while paying the agricultural producers who remove CO₂. The monitoring system disclosed herein provides the sequestered carbon detection, quantification, registration and tracking data technical infrastructure needed to support all the financial mechanisms, including insurance, necessary to promote growth and investment for agricultural producers, and other carbon dioxide sequestration providers, who sequester carbon dioxide.

Agriculture—specifically small-scale agriculture across farming, ranching, and agroforestry which make up nearly 50% of United States farmland—provides the greatest immediate impact towards atmospheric CO₂ removal. To perform their roles efficiently, small agricultural businesses cannot be hindered by increased demands on their time or activities that do not provide a substantial return on their investment. It is in this regard that scientifically rigorous process of sequestered carbon measurement described herein relies on a combination of any one or more of physical biomass sampling, satellite, drone, and other remote data collection methods, as well as data ingestion from forestry product accounting, or Enterprise Resource Planning (ERP) systems. By reducing the administrative data gathering and reporting requirements on farmers and foresters, they can focus on improving the quality of their products while providing them with ownership of the accurate sequestered carbon assets, without adding to their burden.

The presently disclosed carbon monitoring system provides a method to ingest public and private carbon measurement data along with current physical-state and location of sequestered carbon permanently stored in transportable mediums which include but are not limited to lumber, biochar, and transmuted limestone. By tracking the entire life cycle of sequestered carbon, from atmospheric removal until re-release, sequestered carbon asset data, and associated registry data provide a level of data accuracy and integrity not currently available. Long term, and accurate sequestered carbon measurement is the only way to ensure that carbon removed from the atmosphere stays sequestered, regardless of the storage medium. Additionally, quantitative measurement and data integrity provided by the monitoring system disclosed supports growth of carbon asset trading as an economic vehicle to drive long term greenhouse gas reductions.

The carbon monitoring system disclosed herein comprises multiple computational systems which include a continuous monitoring engine, a registration database, a transactional digital ledger, and a carbon continuous settlement engine. Each computational system supports unique mechanisms, which use one or more structured transformation models (STMs) specified in computer object code, representing both Gaussian and logical transforms that interoperate with other similar mechanisms and computational systems to create an ongoing, self-referencing, self-updating, and continuous state monitoring and recording system for tracking the entire carbon sequestration lifecycle including the re-release of sequestered carbon.

Each unique mechanism in the carbon monitoring system disclosed herein functions as a model-based state engine. Specifically, a continuous and automated system that can receive input objects from one or more data sources—which include by non-limiting example, data collection sensors, a structured data input, or output from a separate model-based state engine—and performs interpretation, analysis, and configuration, via processing circuitry, or code, using configurable on-the-fly modeling objects via a specified model structure. This system and its operational process standardizes disparate data inputs to allow for appropriate transformation based on the mechanism's need. This includes but is not limited to aggregation, mathematical calculations, algorithmic application, data formatting, error resolution, and modality translation.

In certain embodiments, such as mechanisms which receive data streams of non-uniform data objects and/or data objects from multiple sources the model-based state engine performs data interpretations. The engine maintains a configurable object array, which includes all allowable object types along with taxonomic structure outlining objects including but not limited to: data objects, code objects, manipulation objects and processes, and output formatting structures.

By way of example, for data objects, in some embodiments the taxonomy specifies the data type, location, access protocol, descriptive information, set structure types, and format of the object. For code objects, in some embodiments the taxonomy specifies: location of the object or application programming interface (API), access protocol for a driver or API, descriptive information that can include name, free text description, and user documentation, and a language, such as a programming language or API documentation. For manipulation objects, in some embodiments the taxonomy specifies: an algorithm, the data object for application, checksum or error reduction method, and output formatting.

A runtime execution of that previously described, exemplary data is performed by the processing circuitry of a server, which includes one or more processors coupled to one or more computer-readable, non-transitory storage media, with the media including therein instructions to operate the one or more processors, to cause the server to run one or more software applications. The software application invokes the code objects appropriate for the data processing via a uniform resource locator (URL). In certain embodiments the invoking application accesses information across one or more data objects. When the execution is complete, the output information is recorded and processed in accordance with the manipulation objects requirements.

During the execution, one or more applications may occur in parallel across one or more individual computing nodes. When a node has completed processing based on one or more logic steps, control of the object and the resulting output may be transferred to one or more nodes based on required outcomes. In some embodiments, the outcome is a choice that is selected by the node based on internal logic steps of the node or user input to the node. In addition, control can be passed directly to another node. In some implementations, a node may depend on one or more data outputs from at least one previous node so the node may need to wait for the at least one previous node to execute before commencing execution.

As would be understood by one of ordinary skill in the art, based on the teachings herein, in some embodiments of the monitoring system disclosed herein, a mobile device or external computational device is used in the same manner as the server to receive sensor or data inputs, send the data to the continuous monitoring engine and the engine would normalize and interpret the inputs against an input standardization model, and/or a legal conformance model, and/or a life-cycle/physical state change model, and/or a settlement model. All processes within each model-based state engine, and communication between independent state engines, are performed automatically with the server, without human intervention, based upon the pre-configured object rules.

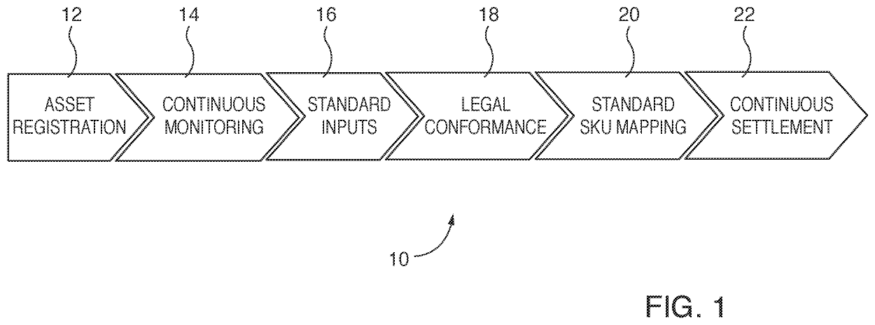

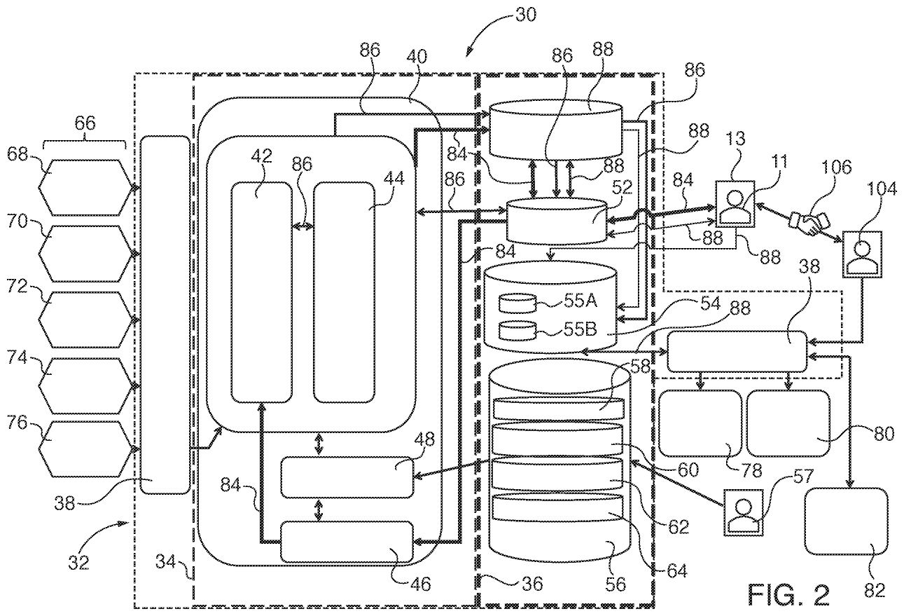

FIG. 1 is a lifecycle flow chart of the greenhouse gas emission and sequestration monitoring process 10 practiced by the monitoring system 30 of FIG. 2. The carbon monitoring system 30 implements process functions for: 1) registration of carbon sequestering assets; 2) collection and integration of continuous monitoring inputs; 3) standardization of continuous monitoring inputs using a standardization model, 4) application of a legal conformance model to render a given set of measurements for a given asset into a legal credit/offset unit for a given jurisdiction; 5) a SKU mapping database that allows for mapping of a given asset-measurement-conformance setting to a SKU in a participant accounting or ERP system; and 5) implementation of the conformed continuous asset monitoring with smart contracts to support continuous settlement.

As a general overview of how the carbon monitoring system 30 implements the monitoring process functions 10 of FIG. 1, it translates continuous monitoring inputs into continuous settlement on registered carbon assets upon state changes, using a hierarchical set of embedded models, comprising a domain specific state transition model that specifies which measurements are applied to which carbon sequestering assets and what legal credits or offsets are generated at each state. The models are hierarchical and specified in JavaScript Object Notation (JSON), and reuse general measurement standardization models (e.g., conformance of soil monitoring outputs for different sampling kits or conformance of satellite spectroscopy images), reuse general legal conformance models that are jurisdiction specific and rely upon the standardized measurement outputs, and use a domain specific SKU mapping model to apply the asset-specific legally conformed measurements to the appropriate state in a domain lifecycle model. The result is the carbon monitoring system 30 described herein reliably tracks the flow of sequestered carbon, and maintains a continuous, verifiable, dynamic ledger of the sequestered carbon, via feedback loops through a model-based computing system, whose architecture is shown in FIG. 2. This model-based system allows for n-tiers of descriptive models for standardization, conformance, and state tracking and transition to support automated carbon settlement through continuous monitoring and feedback loops integrated with a digital ledger. The carbon monitoring system 30 uses a message/queue driven architecture to monitor continuously registered assets for carbon content and state change, supported by interpretable JSON models that provide on-the-fly logic for measurement standardization, legal conformance, determinants of state change, and mapping to SKUs for use in remote accounting and ERP systems.

More particularly, referring to both FIGS. 1 and 2, a carbon asset owner 11 and/or its designated third party (RAO) initiates registration 12 of one or more sequestered carbon assets at one or more locations via a user communications device 13. In response to a monitoring system query, the RAO 11 provides initial baseline data about the sequestered carbon assets, including location, type, quantity, and ownership. The system continuously monitors new asset data concerning the registered carbon assets, provided by the RAO, or designated third parties 12, and converts inputted the carbon data into standard input 16, asset data streams (ADS) that are in legal conformance 18 with governmental requirements for registration of carbon credits. The monitoring system 30 generates and maps stock keeping units (SKUs) 20 to track material transformation and/or transport the sequestered carbon in the stream of commerce and provides continuous settlement 22 of carbon sequestration asset units (SCAUs) linked to each ADS. An SCAU is the tradable and transferrable unit of carbon, functionally equivalent to a unit of currency or a share of stock. The SCAUs are accessible to authorized users of the monitoring system 30, including governmental regulatory authorities, scientists and other environmental researchers, financial services companies, RAOs, and financial intermediaries who trade carbon credits.

General architecture of the monitoring system 30 is shown in FIG. 2; it is implemented on a computer server 32 including one or more processors 34 coupled to one or more computer-readable, non-transitory storage media 36. The storage media 36 include therein non-transitory instructions, including instructions in computer programs, to operate, when executed by the one or more processors, to cause the server 32 to perform carbon data monitoring tasks further described herein. While there is reference to an exemplary server 32 platform architecture with implementation by software in the storage media 36, in other embodiments the monitoring system 30 is implemented in various forms of hardware, software, firmware, special purpose processors, or a combination thereof. The server 32 platform also includes an operating system and microinstruction code. The various processes and functions described herein are either part of the microinstruction code or part of the program (or combination thereof) which is executed via the operating system. In addition, various other peripheral devices and remote communications pathways are connected to the server 32 by service interface bus 38.

In the embodiment of FIG. 2, the server 34 executes a continuous monitoring engine (CME) 40 to coordinate overall operation of the monitoring system 30, including operation of other modeling engines. Each computational system, including modeling engines running therein support unique computational mechanisms, which use one or more structured transformation models (STMs) specified in computer object code, representing both Gaussian and logical transforms that interoperate with other similar mechanisms and computational systems to create an ongoing, self-referencing, self-updating, and continuous state monitoring and recording system for tracking the entire carbon sequestration lifecycle including the re-release of sequestered carbon. The CME 40 executes an extract, transform and load (ETL) and processing module 42 that extracts various input data from its original source format, and transforms the data (e.g., to ADS formatting rules), for example with one or more STMs. The CME 40 loads the transformed data to a target database, such as the reference metadata store 46, and processes that data (e.g., to calculate quantity of sequestered carbon in an inputted data sample) in response to event-management messages and topics queues 44. The processing module 42 specifies and executes models obtained from the model loader and interpreter 48 to process the data. The STMs are stored in model storage unit 56 as self-referencing data objects that expose input-output metadata, such that a model-based interpreter 48 can automatically load the relevant STMs for a given set of data for a combination of ADS and SCAU, such that the interpreter will read the stored data objects and perform Gaussian and logical transforms at runtime.

The CME 40 is coupled to a digital ledger 50; the latter stores carbon sequestration asset units (SCAUs) linked to each ADS, on behalf of the sequestering RAO 11. When required for the ADS, the CME 40 assigns one or more stock keeping units (SKU) to sub-units of the sequestered carbon to monitor material transformation and/or transport thereof in the stream of commerce and communicates the SKU data as well as the SKU data to the RAO 11, via the latter's user communication device 13. SCAUs are data subsets of the corresponding ADSs, but they are independent units.

The CME 40 and the digital ledger 50 are coupled to a registration database 52. The registration database 52 stores the plural ADS, each of which includes identity of the RAO 11 or its designated third party (RAO identifier), a geographic location identifier, and carbon content specifications required to record physical characteristics and quantity of sequestered carbon. RAO access credentials for accessing the server are also stored in the registration database 52.

The registration database 52 generates a new ADS therein, in response a user request to become an RAO 11. The user request is communicated to a portal of the server 32, via an application programming interface (API) running on a communication device 13 of the user; for example, a personal computer, tablet computer, smart phone, or a remote server running enterprise resource planning ERP software. In some embodiments, the user's communication device 13 API utilizes JavaScript Object Notation (JSON) data-interchange format, in communication with the service interface bus 38 of the server 32 of the monitoring system 30. User registration information inputted to the registration database 52 is stored in JSON format. Exemplary information inputted to the registration database 52 by the RAO 11 are in Table 1.

TABLE 1

| | | Data | |
|---|---|---|---|
| Item | Description | Structure | Data Type |
| Name | Business | String | String |
| | Entity Name | | |
| Parcel Set | Location of | Array | String |
| | each land | | |
| | parcel or | | |
| | unique | | |
| | sequestration | | |
| | location | | |
| Sequestration | Sequestration | Array | String |
| Method | method(s) | | |
| | associated | | |
| | with each | | |
| | individual | | |
| | parcel location | | |
| Input Set | Describes set | Array | String |
| | of inputs | | |
| Output Set | Describes set | Array | String |
| | of outputs | | |
| Normalization | Describes the | Array, string | |
| Factor | calculations | | |
| | required to | | |
| | normalize | | |
| | outputs | | |
| Ownership | Verification of | Array | String, |
| Identification | ownership | | blob, int |

In processing the user request, the registration database 52 automatically generates a business entity unique identifier and synchs it across the CME 40 and the digital ledger 50. Each parcel or location has a unique parcel identifier automatically generated in the registration database 52 and it is synchronized to the business unique ID. Methods of sequestration are also linked to each unique location ID based on user inputs during registration. Each method of sequestration requires specific data collection; the relevant data collection requirements are sent to the CME 40, as well as RAO access credentials.

In response to the user request, the server 32 sends the RAO access credentials to the user and now recognizes the user as an RAO 11. In simpler embodiments, the communication device is a smart phone or tablet executing an API. In larger enterprises the RAO's ERP data system communicates with the registration database 52 to transfer data to the server 32 of the monitoring system 30.

The digital ledger 50 is coupled to a carbon continuous settlement engine 54 that facilitates trading of sequestered carbon credits associated with the SCAU data in the digital ledger. The continuous settlement engine 54 functions as a digital wallet repository, aggregating individual wallets, such as the wallet 55A of an RAO 11 and the wallet 55B of an exemplary carbon asset purchaser 104. The digital ledger 50 modifies the SCAU data to reflect changes in ownership of and/or offsetting consumption of sequestered carbon and communicates same to the continuous settlement engine 54. The carbon continuous settlement engine 54 is alternatively referred to herein as a digital wallet.

Model repository 56 stores models that are utilized by the CME 40 when operating the monitoring system 30. Domain experts 57 update the stored models. Exemplary models stored in the model repository 56 include, but re not limited to domain lifecycle models 58, legal conformance models 60, measurement standardization models 60, and SKU mapping models 62. The models are loaded into the model loader and interpreter 48. The models are stored in a common JavaScript Object Notation (JSON) data-interchange format.

The CME 40 receives input sensor data 66 relevant to plural ADS via the service interface bus 38. Input sensor data 66 include, but are not limited to any of: land registry (local GIS) data 68, agricultural crop management data 70, satellite observational data (e.g., NASA Landsat) 72, transactional data from ERP systems of third parties (e.g., electric power, fuel, fertilizer, construction materials consumption of an RAO 11 or insurance company loss reports of sequestered carbon release attributable to a burned down building owned by an RAO), or site monitoring sensors 76 (e.g., measuring captured carbon concentrations in storage vessels, carbon composition of soil samples, tonnage of harvested crops).

The server 32 of the monitoring system 30 is in remote communication with third parties, via its service interface bus 38. Such third parties include, by way of nonlimiting example financial intermediaries 78 monitoring SCAU trades generated by the continuous settlement engine 54, government regulators receiving carbon emission compliance reports 80, other data users 82 who monitor carbon emission and extraction trends, such as environmental scientists, and carbon asset buyers 104 who have executed carbon trades 106 with RAO's 11.

There are three principal data flow and feedback loops for ADS and other data in the server 32. Feedback loop 84 comprises data flow among the CME 40, the digital ledger 50, the registration database 52 and the user communication device 13 of the RAO 11. Feedback loop 86 comprises data flow among the CME 40, the digital ledger 50, the registration database 52, and the continuous settlement engine 54. Feedback loop 88 comprises data flow among the digital ledger 50, the registration database 52, the continuous settlement engine 54 and remote third parties (e.g., RAO communication device 13, asset buyers 104, financial intermediaries 78 governmental compliance report recipients 80), via the service interface bus 38.

In feedback loop 84, the RAO 11 registers a land parcel in the registration database 52 details the method(s) of sequestration occurring at each location or land parcel (example: location 1 is just direct air carbon capture, location 2 is just ranching, location 3 is timber production which combines timber and soil measurements). The registration database 52 is the user facing portal for accessing the majority of visually accessible information aggregated from the entire carbon monitoring system 30. Reporting, account balances, carbon asset transfers, and other transactional communications with the RAO 11 regarding carbon data are performed in feedback loop 84. After the RAO 11 initiates the parcel registration process, bi-directional communications with the RAO's ERP system allows that system to ingest sequestration location account balances from the monitoring system 30, and for registration database 52 to ingest ERP data from the RAO for passing onto the CME 40. The registration database 52 passes the RAO information, including location sequestration method and other details onto CME 40. Based on the parcel information data received from the RAO 11, the CME 40 determines the required baseline measurements necessary for each sequestration method. The CME 40 establishes and stores a new ADS for each newly registered location/parcel in the registration database 52. The registration database 52 sends RAO information to digital ledger 50, to establish a ledger account for RAO 11. The digital ledger 50 establishes a parallel, new ADS for the RAO 11, indicating the newly registered location/parcels, which CME 40 will update in the future. The CME 40 processes changes to ADS data, using the stored models in the model repository 56. For a newly established ADS, the CME 40 requests a baseline data set from the ERP system of the RAO 11, which the latter submits automatically via its registration database credentials. In situations where baseline data is supplied by a third-party data collector (e.g., a soil sample analysis lab or a forest surveyor), the CME 40 generates and sends a data collection notice to the third party and the latter responds through a portal of the server 32.

Periodic data updates for existing ADS are processed by the CME 40 in feedback loop 84. Remote sensing data, such as satellite data 74 or drone monitoring data, are collected continuously and globally in recurrent fashion. When a location is initially registered, the geo-location data from the satellite path is requested from the satellite data collection system and ingested by the CME 40, where it is associated with any manual collected data sets already in the ADS. This activity provides intra-manual collection data, and uses manual collected data to calibrate the satellite datasets 74, while also improving the self-updating requirements of the CME 40. The CME 40 periodically sends manual data submission request notices to the RAO 11 and any specified ones of the sensor data 66 submitters. As was done with the initial data set submission requests, a third-party data collector (e.g., a soil sample analysis lab or a forest surveyor responds by sending the updated requested data through a portal of the server 32.

In the feedback loop 86, between the CME 40 and the digital ledger 50, the provides updated ADS data to the digital ledger, detailing increase or decrease on sequestered carbon volume at each RAO's location/parcel, and sub location detail information if necessary (such as individual tree IDs and sequestration volume per tree). The digital ledger 50 in turn updates its corresponding ADS data regarding carbon balance information at every relevant location and sub-location for each RAO 11. This digital ledger 50 updating process includes the CME 40 filtering and association of regulatory restrictions to tag each location's resulting measured sequestration volumes as SCAUs by appropriate legal standards set by the legal conformance models 60. Each SCAU is tagged with the various regulatory body approvals to allow matching for subsequent carbon trading in the continuous settlement engine 54. The CME 40 passes to the digital ledger 50 all changes of sequester carbon state and location. Thereafter the digital ledger 50 passes updated RAO 11 location account balances back to the registration database 52. The digital ledger 50 also passes updated SCAU data (including regulatory approval tags) to the digital wallet 55A of the RAO 11 in the continuous settlement engine 54, which facilitates trading of the RAO's carbon assets.

Feedback loop 88 facilitates trading and self-effectuating correction or adjustment of carbon assets. Coupling of the digital ledger 50 and the digital wallets of the continuous settlement engine 54 allows an RAO 11 to trade its SCAUs directly with potential asset buyers 104 or through intermediary, established carbon-trading markets and exchanges. In the case of direct trades 106, the parties negotiate sale outside of the monitoring system 30; the RAO 11 authorizes direct transfer of the SCAUs to the buyer 104, through a transfer notice to the registration database 52, via its user communication device 13. The registration database 52 passes the transfer authorization to the continuous settlement engine 54, which in turn transfers the relevant SCAUs from the RAO's digital wallet 55A to the buyer's digital wallet 55B. The continuous settlement engine 54 flags and isolates the transferred SCAUs pending receipt of purchase funds transfer confirmation. Where an RAO 11 lists carbon assets for trade on a market or exchange, the RAO links its digital wallet 55A with external brokerage or exchange, which may go through a clearing house or allow self-clearing, and automated settlement system outside of the monitoring system 30. The RAO 11 or its broker lists the RAO's SCAUs in the digital wallet 55A for trade and sale. After the trade occurs, the traded SCAUs are withdrawn from the RAO's digital wallet 55A and transferred to the digital wallet 55B of the buyer 104. The buyer 104 accesses its purchased SCAUs through the registration portal 52. It is noted that sold or otherwise transferred SCAUs continue to be linked to the RAO 11 for updates from the digital ledger, separate from the change of ownership to the buyer 104.

In feedback loop 88, where a buyer 104 retires a purchased SCAU as a credit against its own generated carbon emissions (e.g., a power generation or manufacturing business exceeding its legal emission limits), the continuous settlement engine 54 marks the subject SCAU as “retired” but it continues to be linked to the selling RAO for updates from the digital ledger 50. The continuous settlement engine 54 will not allow retired SCAUs to be sold.

In feedback loop 88, when the CME 40 determines that there was a re-release of sequestered carbon associated with an SCAU into the environment, it notifies the digital ledger 50 and the latter categorizes the carbon loss for further processing of the associated SCAU. If the relevant ADS is registered as having insurance coverage, the digital ledger 50 causes a loss notification to be sent to the registered insurance company and places an order on a carbon market/exchange to purchase replacement SCAUs to mitigate the loss. The replacement SCAUs are transferred to the insured RAO's digital wallet 55A. The replacement SCAUs continue to be tracked back to the selling RAO 11 for long term tracking. In the event of an uninsured loss, the digital ledger 50 checks to see whether the loss RAO 11 has sufficient positive balance in its digital wallet 55A to cover the loss for any planned or existing trades linked to its digital wallet. If there is a sufficient balance, the digital ledger 50 and the RAO's digital wallet 55A are reduced by the loss and loss notice is provided to the registration database 52 to update the associated ADS. If there is an insufficient balance, the digital ledger 50 triggers a correction event and notifies RAO 11 through registration database 52 of permitted time to rectify the loss event before the monitoring system 30 purchases replacement SCAUs on its behalf.

Summarizing the described functions performed by the CME 40, directly or through intermediary modeling engines previously described, for each ADS the CME upon initial registration of a new ADS, defines initial baseline data needed to quantify the physical characteristics and quantity of sequestered carbon at the registered location. The CME 40 communicates a request for the defined initial baseline data to and receives them from the RAO 11, via the latter's user communication device 13, and conforms the received initial baseline data to its internal data interchange format. The CME 40 periodically communicates with the RAO 11, via user communication device 13, to receive and collect new data for an existing ADS, including any changes in any one or more of: quantity of sequestered carbon attributable to physical processes of additional sequestration from and/or release of either previously sequestered or new carbon sources into the environment, and/or transformation of physical properties, and/or transport into or away from the registered sequestration location and/or ownership thereof. The CME 40 conforms the newly received data to its internal data interchange format and updates the ADS. The CME 40 also communicates with the RAO 11, via the user communication device 13, to request new data to update an existing ADS to conform with modification of ADS specifications or requirements, such as in response to changes in carbon emission regulations of a governmental authority in the legal conformance model 60, conforms the newly received data to its internal data interchange format, and updates the ADS. When required, the CME 40 assigns one or more stock keeping units (SKU) to sub-units of the sequestered carbon in an ADS to monitor material transformation and/or transport thereof in stream of commerce and communicates the SKU data to the RAO 11, via its user communication device 13.

The CME 40 generates carbon sequestration asset units (SCAUs), linked to each ADS, wherein each SCAU includes origination location and current location of sequestered carbon. The CME 40 communicates with external data services, including both sensors and external computer accounting systems, associated with specific ADS in the registration database, to collect component data from the RAO and third parties in native format. The CME 40 transforms data throughout the continuous monitoring process using a model-based interpreter that uses one or more structured transformation models (STMs) specified in object code representing both Gaussian and logical transforms. The CME 40 stores STMs as self-referencing data objects that expose input-output metadata such that the model-based interpreter can automatically load the relevant STMs for a given set of data for a combination of ADS and SCAU such that the interpreter will read the stored data objects and perform Gaussian and logical transforms at runtime. The CME 40 specifies STMs that perform Gaussian and logical transformations on the input data to support physical property calculations, legal compliance tests, and accounting allocations. The CME 40 transforms ADS component data to normalized elements for aggregation and analysis using a model-based interpreter that specifies normalizations specific to data input source and data elements. The CME 40 aggregates and analyzes plural ADS, SKU and SCAU data to derive any temporal changes in ownership, quantity and/or physical properties of sequestered carbon attributable to: additional sequestration from, and/or release of previously sequestered carbon, and/or introduction of new carbon sources into the environment, transformation of physical properties of the sequestered carbon, and/or transport of sequestered carbon into or away from the registered sequestration location, using applied STMs in the model-based interpreter. The CME 40 updates each ADS, SKU and SCAU data in a continuous feedback loop.

It is to be understood that, because some of the constituent monitoring system 30 components, modeling engines, and method steps depicted in the accompanying figures are preferably implemented in software, the actual connections between the system components (or the process steps) may differ depending upon the manner in which the monitoring system is programmed. Specifically, any of the computer platforms or devices may be interconnected using any existing or later-discovered networking technology and may also all be connected through a larger network system, such as a corporate network, metropolitan network, or a global network, such as the Internet.

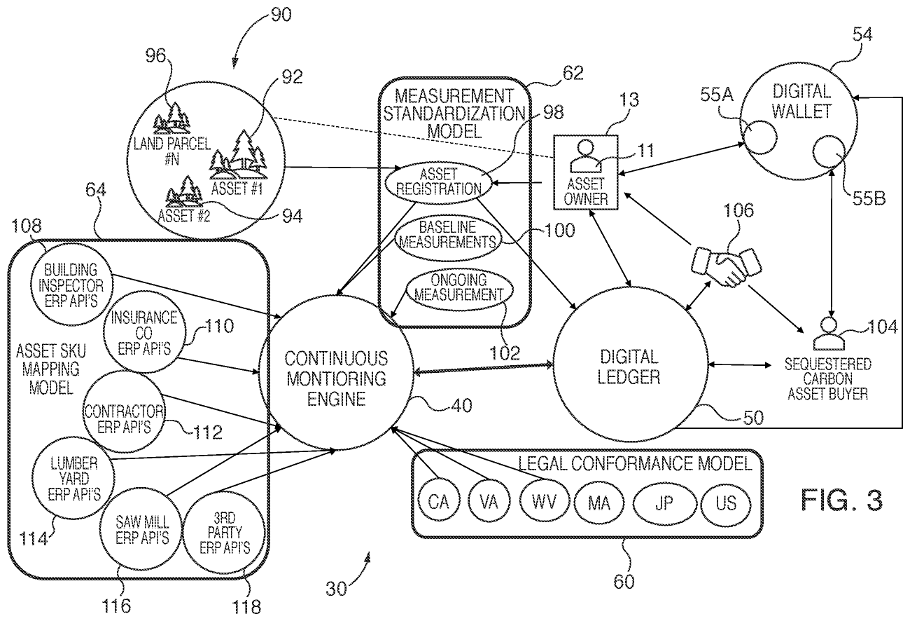

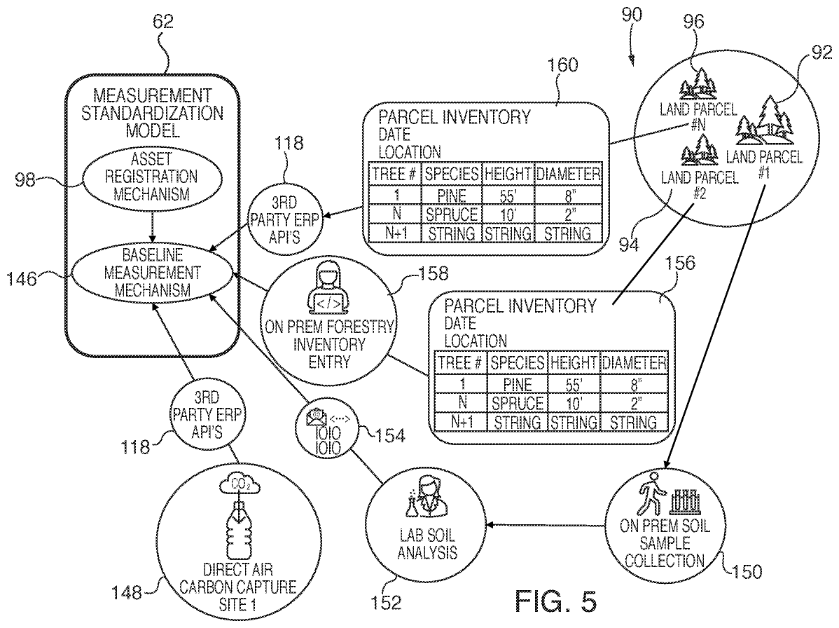

FIG. 3 illustrates functional interaction of the models executed in the CME 40 the digital ledger 50 and the continuous settlement engine (i.e., digital wallet) 54. The CME 40 executes the measurement standardization model (MSM) 62, based on asset registration 98 requested by an RAO 11, via its user communication device 13. Referring to FIGS. 3 and 5, during asset registration, the RO 11 inputs land parcel data 90, which as shown comprises separate groups of carbon-sequestering forest assets 92, 94 and 96. The MSM 62 acquires baseline measurements 100 by a baseline measurement mechanism 146, to create an initial ADS for each of the separate assets 92, 94, 96.

The baseline measurement mechanism 146 is tailored to receive plural types of asset data inputs. In FIG. 5, land parcel 92 sequesters carbon in soil. An on-premises soil sample is collected 150 and sent to a soil laboratory 152 for analysis of carbon that is sequestered in the soil. The soil laboratory submits the analysis data 154 to the CME 40 and forwards same to the MSM 62. Subsequently the MSM 62 updates the respective ADSs associated with land parcel 92 with new ongoing measurement data 102.

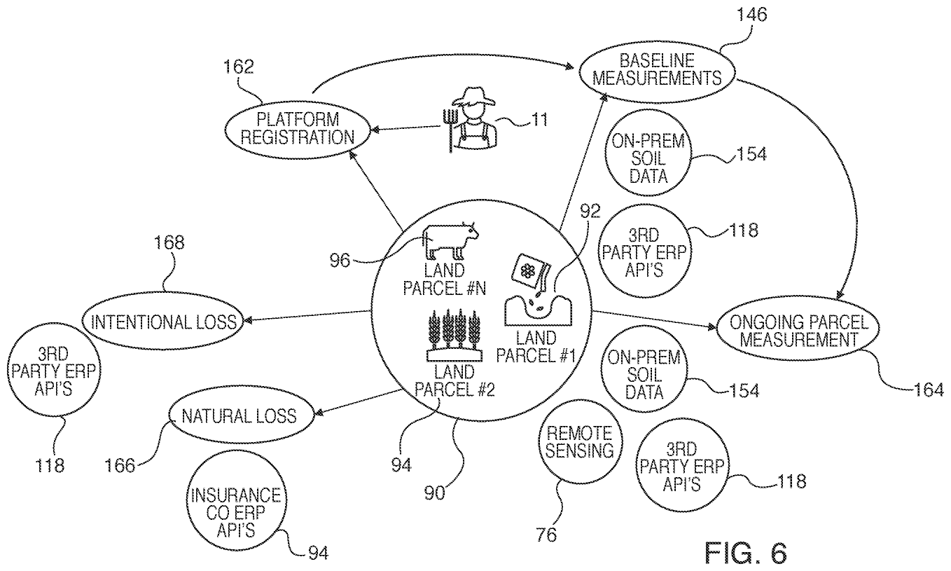

In the soil-based, carbon measurement process of FIG. 6 core samples 154 are collected at multiple locations on a specific land parcel 90. Sampling is performed every 6″ or 15 cm down to a depth of 6 ft or 180 cm. Measurement units are recorded at time of sample collection. Each sub-sample is kept separate, given a unique ID, and identified with what layer of the core sample it occurred. Latitude/longitude position of each core sample is recorded and tagged to each sub-sample, along with its depth. Collected soil samples are packaged and transferred to the soil laboratory for analysis. Sub-sample IDs and associated geo-location, depth location, and date of collection are transmitted to the CME 40 and held in a pending state. Lab analysis of the soil samples uses a method of high-temperature combustion to measure the Total Organic Carbon content volume of each sub-sample. Total Organic Carbon content volumes for each sub-sample are transmitted to the CME 40. Lab result data is associated with the pending state sub-sample IDs. The model-based, soil-sampling state engine applies an algorithm based on input from soil carbon experts, known pedometric science, and recursive learnings to determine the volume of Total Organic Carbon measured at each sub-sample depth for the specific parcel associated with the different samples. These sub layer totals are combined further for the specific parcel and calculated in a Tons of CO₂ equivalent.

First time measurements create a baseline value upon which future measurements are compared. Increases in Total Organic Carbon reflect an increase in actual sequestered carbon. Increases in actual sequestered carbon generates Sequestered Carbon Asset Units, through the combination of continuous measurement and digital ledger functions.

Land parcel 94 sequesters carbon in timber. A parcel inventory 156 for land parcel comprises each individual tree, its species, and its diameter. Each tree is given a unique tree ID within the CME, and that tree ID is tied to the forestry inventory ID. Each tree ID is linked to the parcel ID and the registered business ID. The parcel inventory 156 is transmitted to the CME 40/MSM 62 by manual data entry 158 on a personal computer, communicating through an API. Land parcel 96 also sequesters carbon in timber; its parcel inventory 160 format is the same as parcel inventory 156. Parcel inventory 160 is transmitted to the CME 40/MSM 62 via an ERP/API 118 of the RAO 11 or its authorized third-party representative.

The MSM 62 in FIG. 5 also receives carbon data from a direct-air carbon capture site 148, transmitted via an ERP/API 118 of the RAO 11 or its authorized third-party representative. Where carbon is captured in gaseous form, the baseline data include current volume and pressure and the current grid electrical meter reading at the time of location registration. Where carbon is captured by transmutation storage, the baseline data include current volume of transmuted carbon storage on site and the current grid electrical meter reading at the time of location registration. In subsequent updated data submissions, the CME 40 will determine net change in direct-air captured carbon by subtracting carbon attributable to electric consumption from the sequestered carbon inventory.

The CME 40 executes the legal conformance model 60, which includes carbon emission and sequestration regulations for plural governmental entities (e.g., Japan, the United States of America, and states within the United States, including without limitation California, Virginia, West Virginia, and Massachusetts). The legal conformance model 60 enables the CME 40 to tag each ADS with information including whether the sequestered carbon represented therein meets carbon-emission offset legal requirements of various jurisdictions.

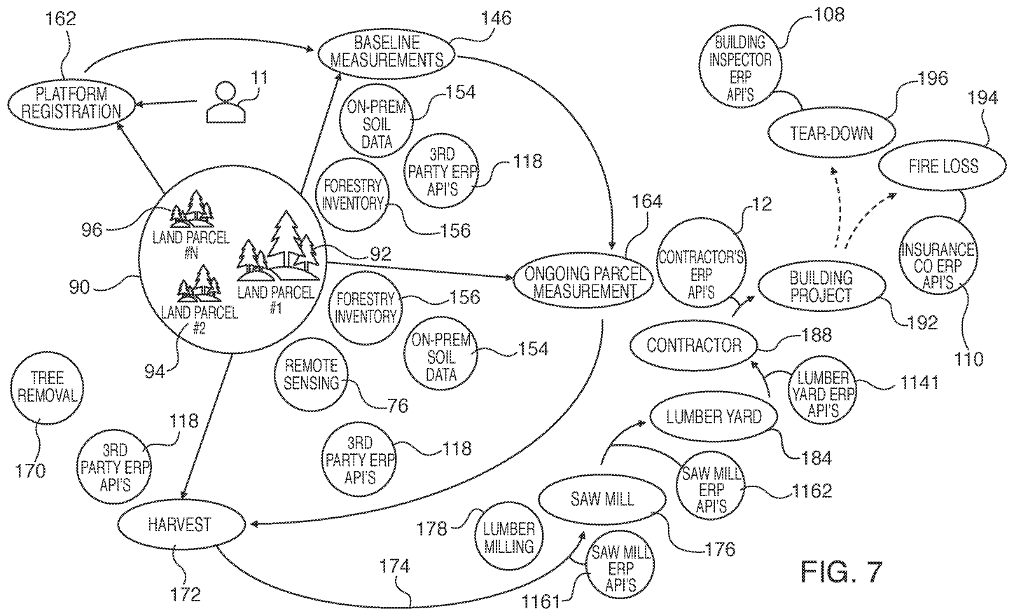

The CME 40 executes the asset SKU mapping model 64 for each ADS to identify the initial state and temporal change of state of sequestered carbon, including quantity, physical characteristics, geographic location, transformation into other materials and transportation. The SKU mapping model 64 relies on sensor inputs and reporting by various entities in the stream of commerce, which by way of example for forestry products (see FIG. 7) include building inspectors 108, insurance companies 112, building contractors 112, lumber yards 114, sawmill operators 116, and other third parties 118, such as soil sample labs. In some embodiments the sensor inputs to the asset SKU mapping model 64 are communicated by APIs in the reporting entity's ERP systems, but in other embodiments the reporters send manually imputed data through APIs of personal computers, tablet computers and smart phones.

The asset SKU mapping model 64 is a compilation of multiple sub-mechanisms corresponding to unique datasets and/or providers for various stages and states of materials and goods containing sequestered carbon in the stream of commerce.

The digital ledger 50 receives the plural ADSs from the CME 40 that were generated through use of the MSM 62 and the asset SKU mapping model 64. It tracks state changes of each ADS, SKU and SCAU. When an RAO 11 and a buyer 104 agree to a trade 106, they communicate the trade event to the digital ledger 50. The ledger 50 in turn records the trade and causes the monitoring system 30 to update the ADS, SKU and SCAU data to reflect the trade. The digital ledger 50 also notifies the continuous settlement engine 54 to post the trade to the respective digital wallets 55A and 55B of the RAO 11 and the buyer 104.

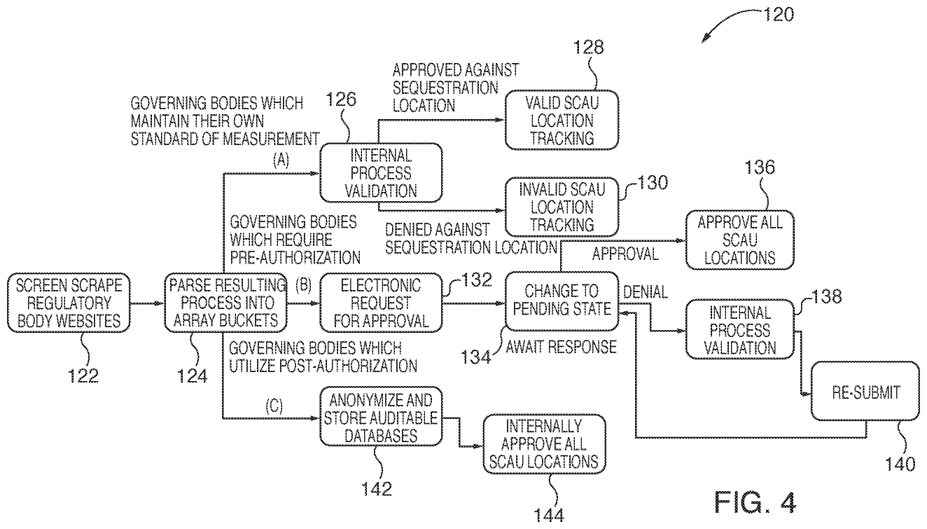

Referring to FIGS. 2 and 4, the legal conformance model 60 executed by the CME 40 of the monitoring system 30 is implemented in a legal conformance state engine 120. In the embodiment of FIG. 4, the conformance state engine 120 applies an html based ‘screen scraper’ 122 to an array of pre-determined state, federal, and international environmental governing bodies to identify processes and procedures for application of sequestered carbon measurement standards for the generation of carbon credits, offsets, or other ‘sequestered carbon asset’ nomenclature. Each governing body is linked to a unique ID and stored in an array 124. The scraped text is filtered through an OCR mechanism to parse text into a machine-readable format. The parsed text is reviewed to determine methods and procedures of how the governing body measures sequestered carbon content. Particularly, whether the governing body: (A) maintains its own standard of measurement, (B) must pre-authorize new methods of measurement, or (C) requires post authorization of new methods of measurement. The legal conformance status engine 120 associates each governing body's identification code with its method or procedure (A), (B) or (C).

For governing bodies which maintain their own standard of measurement (A), the conformance status engine 120 compares the prescribed standard of measurement against the data sets and measurement methods for each specific land parcel or sequestration location to validate the site's activities against the regulatory standard at step 126. Those sequestration locations and/or and parcels which fall within the prescribed standard (A), at step 128 the status engine 120 links the appropriate IDs for each governing body, demarcating the CME 40 tracked SCAUs as being validated for the governing body's carbon credit, or other asset nomenclature. If a site does not meet the requirements, it is not eligible for the specific regulatory body, however it is still tracked as an SCAU within the CME 40.

For governing bodies which require pre-authorization (B), at step 132 the CME 40 shall transmit an email to the contact scraped from the governing bodies website, describing in human readable terms, the processes used for calculating the generation of SCAUs. Until confirmation of pre-approval is transmitted back to the CME 40, each regulatory body will be linked to the tracked location or site as ‘pending’ at step 134. Upon pre-approval, all associated sites and resulting SCAUs will become associated with the regulatory body and tracked as the appropriate nomenclature for that body's carbon credit or SCAU at step 136. If denied, details of the denial trigger an analysis of process to identify missing measurement activities at step 138; thereafter at step 140 the conformance status engine 120 evaluates whether to resubmit the electronic request with updated measurement processes or challenge the denial.

For governing bodies which utilize post authorization (C), measurement and anonymized audit data will be stored in human readable format for review at step 142. If an SCAU's data structure meets or exceeds the requirements for a specific set of requirements, at step 144, both the SCAU and its related dataset within the CME 40 are tagged with the compliance code for the requirement set (i.e., individual state requirements vs federal requirements) to allow for future auditability of each individual SCAU or derivative carbon credit.

FIG. 6 illustrates the carbon state monitoring model utilized in the CME 40 for measuring carbon sequestered in soil. An RAO 11 is registering three different carbon assets 92, 94, 96 located on land parcel 90 during platform registration 162. The CME 40 requests baseline measurement data 146 and receives on-premises soil data 154 via an ERP system of a third party authorized by the RAO 11. After creating an initial ADS for each of the carbon assets 92, 94, 96 the CME 40 schedules ongoing parcel measurements 164 for additional on-premises soil data 154 and by remote sensors 76, which are submitted to the CME by third party ERP systems 118. In the event of a natural loss event 166, an insurance company 110 that covers the loss submits a loss report to the CME 40 via its ERP system. The carbon state monitoring model deducts the carbon loss from its associated ADS, resulting in a net carbon loss for the associated asset 92, 94 or 96. For example, fuel volumes for transport of soil samples, grid electricity volumes, and carbon content released during measurement of organic carbon volume measured in each sample is passed to the CME 40 for carbon loss deduction.

Similarly, intentional losses 168, for example attributable to sale of harvested crops, consumption of fossil fuel, electricity, and non-organic fertilizer to grow the associated carbon, are deducted from the relevant ADS.

FIG. 7 illustrates a timber carbon-state monitoring model, and its ability to track transmutation of trees to lumber, and in turn to a completed building that incorporates the lumber, and transportation of lumber products to different geographic locations in the stream of commerce. A forestry-land owner/agent RAO 11 registers its land parcel 90, comprising three carbon-sequestering timber assets 92, 94 and 96, on registration platform 162. Each asset in the land parcel receives an identifier code for long term tracking. In response to a request for baseline measurements 146 from the monitoring system 30, the RAO 11 submits on-premises soil data 154, which evidences carbon sequestered in the soil, and forestry inventory of trees 156, which evidences carbon sequestered in timber, via an authorized third party's ERP system 118. In exemplary embodiments, authorized third parties include soil sample laboratories and tree inventory data collectors. At set designated periods or any other time, the monitoring system 30 requests and receives updated, ongoing parcel measurement data 164, e.g., on-premises soil data 154, and forestry inventory of trees 156, from the prior or newly authorized third party's ERP system 118. A third party's ERP system 118 reports a tree harvest 172 to the monitoring system 30. The CME 40 of the monitoring system updates ADS, SKU and SCAU data associated with the harvested timber and tracks transmutation and transport of the timber 174 through the stream of commerce. When the harvested timber 172 is transported to a saw mill 176 for milling into lumber 178, the saw mill's ERP system reports to the monitoring system 30 the timber receipt 1161 and thereafter sale of milled lumber 1162 to lumberyard 184, where thereafter the CME 40 updates ADS, SKU and SCAU data originally associated with the harvested timber to reflect reduction in sequestered carbon attributable to milling waste and consumption of electric power by the saw mill. When the lumber yard 184 sells the lumber to contractor 188 it reports the sale 1141 to the monitoring system 30. The contractor 188 in turn reports with its ERP 112 transfer of the lumber to a building project 192. The monitoring system's CME 40 updates ADS, SKU, and ADS data to reflect changes in the carbon content and location resulting from transmutation of the lumber into a finished building. Over time, the CME updates ADS, SKU, and ADS data to reflect re-release of carbon sequestered in the lumber back into the environment after a building teardown 190 is reported by a building inspector 108 or in case of a fire loss 194, as reported by an insurance company 110.

Exemplary asset SKU mapping model 64 datasets for entities that use forestry products are now described.

A sawmill transforms felled timber into lumber, altering the sequestered carbon quantity. Some of the timber's stored carbon is released back into the environment. Relevant ADS data for a sawmill operation include:
Actual height (length) and diameter of felled timber, associated with individual Tree IDs, and actual weight of felled timber minus limbs and foliage. Combination of both of these ADSs help to improve the algorithm utilized in calculating standing timber carbon storage volumes, to avoid problems of loss tracking once the timber has been cut.
Actual weight and measurement in board feet of lumber associated with individual tree IDs.
Reducing Tree ID carbon volume by off cut and waste material which will biodegrade.
Volume of pulp material by Tree ID
Sales and transfer data of lumber leaving the facility, linked to individual Tree IDs.
Fossil fuel-based energy use in milling, to calculate carbon emissions associated with each tree ID.
Relevant ADS data for a lumber yard include:
Confirmation of purchase volumes of lumber from a Sawmill, by Tree ID.
Sales of lumber to one or more contractors by Tree ID.
Changes to lumber associated with Tree ID, e.g., pre-cutting lumber for sale to customers.
Loss, destruction, breakage
Transport costs to and from the lumber yard, associated with Tree IDs to calculate emissions and reduce carbon storage volumes per Tree ID.
Relevant ADS data for a building contractor include:
Confirmation of purchase volumes from lumber yards.
Association of purchased lumber with a specific building project, including project owner information, the owner's insurance information, and volume of lumber off-cuts (e.g., lumber scrap) not within the envelope of the building project.
Relevant ADS data for an insurance company yard include confirmation of building insurance, current state of a building, and loss reporting.
Relevant ADS data for building inspection include confirmation of existence (e.g., a certificate of occupancy) and demolition notifications.

Although various embodiments that incorporate the invention have been shown and described in detail herein, others can readily devise many other varied embodiments that still incorporate the claimed invention. The invention is not limited in its application to the exemplary embodiment details of construction and the arrangement of components set forth in the description or illustrated in the drawings. The invention is capable of other embodiments and of being practiced or of being conducted in numerous ways. In addition, it is to be understood that the phraseology and terminology used herein is for the purpose of description and should not be regarded as limiting. The use of “including.” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are to be interpreted broadly; they encompass direct and indirect mountings, connections, supports, and couplings. Further, “connected” and “coupled” are not restricted to physical, mechanical, or electrical connections or couplings.

What is claimed is:

1. A sequestered carbon monitoring and data archiving system, comprising:
a server including one or more processors coupled to one or more computer-readable, non-transitory storage media, the media including therein instructions to operate when executed by the one or more processors, to cause the server to:
create and store in the non-transitory storage media in an internal data interchange format, a registration database incorporating: plural asset data sets (ADS), each including: a registered asset owner and/or its designated third party (RAO) identifier, a geographic location identifier, and carbon content specifications required to record physical characteristics and quantity of sequestered carbon; and also incorporating RAO access credentials for accessing the server;
generate a new ADS in the registration database, in response to a user request to become an RAO, the user request communicated to a portal of the server, via an application programming interface (API) or enterprise resource planning ERP software operating on a communication device of the user, the server communicating RAO access credentials to the user and now recognizing the user as an RAO;
operate a continuous monitoring engine, created and stored in the non-transitory storage media, in communication with the registration database, which for each ADS:
upon initial registration of a new ADS, defines initial baseline data needed to quantify the physical characteristics and quantity of sequestered carbon at the registered location;
communicates a request for the defined initial baseline data to and receives them from the RAO or its designated third parties, via its user communication device, and conforms the received initial baseline data to its internal data interchange format;
periodically communicates with the RAO or its designated third parties, via its user communication device, to receive and collect new data for an existing ADS, including any changes in any one or more of: quantity of sequestered carbon attributable to physical processes of additional sequestration from and/or release of either previously sequestered or new carbon sources into the environment, and/or transformation of physical properties, and/or transport into or away from the registered sequestration location and/or ownership thereof, conforms the newly received data to its internal data interchange format, and updates the ADS;
communicates with the RAO or its designated third parties, via its user communication device, requesting new data to update the existing ADS to conform with modification of ADS specifications or requirements used in operation of the continuous monitoring engine, receives the new data from the RAO, conforms the newly received data to its internal data interchange format, and updates the ADS;
when required for the ADS, assigns one or more stock keeping units (SKU) to sub-units of the sequestered carbon to monitor material transformation and/or transport thereof in stream of commerce and communicates the SKU data to the RAO, via its user communication device;
generates carbon sequestration asset units (SCAUs), linked to each ADS, each SCAU including quantity, quality, characteristics, origination location and current location of sequestered carbon;
communicates with external data services, including both sensors and external computer accounting systems, associated with specific ADS in the registration database, to collect component input data in native format;
transforms data throughout the continuous monitoring process using a model-based interpreter that uses one or more structured transformation models (STMs) specified in object code representing both Gaussian and logical transforms;
stores STMs as self-referencing data objects that expose input-output metadata such that the model-based interpreter can automatically load the relevant STMs for a given set of data for a combination of ADS and SCAU such that the interpreter will read the stored data objects and perform Gaussian and logical transforms at runtime;
specifies STMs that perform Gaussian and logical transformations on the input data to support physical property calculations, legal compliance tests, and accounting allocations;
transforms ADS component data to normalized elements for aggregation and analysis using a model-based interpreter that specifies normalizations specific to data input source and data elements;
aggregates and analyzes plural ADS, SKU and SCAU data to derive any temporal changes in ownership, quantity and/or physical properties of sequestered carbon attributable to: additional sequestration from, and/or release of previously sequestered carbon, and/or introduction of new carbon sources into the environment, transformation of physical properties of the sequestered carbon, and/or transport of sequestered carbon into or away from the registered sequestration location using applied STMs in the model-based interpreter;
updates each ADS, SKU and SCAU data in a continuous feedback loop; and
create a digital ledger, in the non-transitory storage media, which is in communication with the continuous monitoring engine, the digital ledger storing the SCAU data in codified data structures that are in compliance with governmental requirements for the generation of tradeable carbon credits, the digital ledger updating, in a continuous feedback loop in conjunction with the continuous monitoring engine, the SCAU data, and the codified data structures for compliance with changes in the governmental requirements.

2. The system of claim 1, further comprising the continuous monitoring engine updating the carbon content specifications of an ADS of a geographic location to: add additional carbon sequestered at or transported into the location, subtract previously sequestered carbon transported to other locations or released into the environment, and subtract additional carbon released into the atmosphere at the location.

3. The system of claim 2, further comprising the continuous monitoring engine updating the carbon content specifications of an ADS attributable to physical transformation of the sequestered carbon into other compositions of matter or other physical forms and creating or revising SKUs associated with the updated ADS.

4. The system of claim 3, further comprising the continuous monitoring engine tagging an SCAU, its related data set, and its related ADS with a mapping to a downstream good represented as a SKU in an accounting system such that tradeable carbon credits can be settled as the good changes hands.

5. The system of claim 4, further comprising the continuous monitoring engine executing runtime-interpreted, object specific Gaussian and logical models for allocation of SCAU credits to different derived, downstream SKUs in the supply chain.

6. The system of claim 1, further comprising: the continuous monitoring engine periodically reinitializes the baseline data definition for an existing ADS, communicates a request to the RAO for new baseline data, updates the ADS with the new baseline data, and updates each SKU and SCAU associated with the updated ADS.

7. The system of claim 1, further comprising the continuous monitoring engine receives reporting data from a third party associated with an RAO's user credentials, concerning ongoing carbon consumption, emission, physical transformation, or transportation associated with an ADS, and updates the ADS and each SKU and SCAU associated therewith.

8. The system of claim 7, the reporting data comprising indirect carbon emission associated with consumption of one or more of electric power, fuel, or products associated with a carbon sequestration process occurring at the location associated with the ADS.

9. The system of claim 8, further comprising the continuous monitoring engine executing runtime-interpreted, object specific Gaussian and logical models for calculation and allocation of direct or indirect carbon sequestration or emission.

10. The system of claim 1, further comprising a user portal coupled to the registration database, and/or the continuous monitoring engine and/or the digital ledger selectively permitting credentialed users access to designated ADS, SKU and SCAU data.

11. The system of claim 1, further comprising the digital ledger in communication with a carbon continuous settlement engine for facilitating trading of sequestered carbon credits associated with the SCAU data in the digital ledger, and the digital ledger modifying SCAU data to reflect changes in ownership of and/or offsetting consumption of sequestered carbon.

12. The system of claim 11, further comprising the continuous monitoring engine tagging an SCAU, its related data set, and its related ADS with a compliance code that it complies with one or more governmental requirements for the generation of tradeable carbon credits.

13. The system of claim 12, further comprising the application of runtime-interpreted, object specific Gaussian and logical models for logical assertion of compliance with a given authority.

14. The system of claim 1, further comprising the continuous monitoring engine tagging an SCAU, its related data set, and its related ADS with a compliance code that it complies with one or more governmental requirements for the generation of tradeable carbon credits.

15. The system of claim 14, further comprising the continuous monitoring engine executing runtime-interpreted, object specific Gaussian and logical models for logical assertion of compliance with a given authority.

16. A method for monitoring and generating asset data sets (ADS) concerning properties and characteristics of sequestered carbon, and archiving same in a database, comprising:
in a server including one or more processors coupled to one or more computer-readable, non-transitory storage media, the media including therein instructions to operate when executed by the one or more processors, the server:
creating and storing in one of its non-transitory storage media, in an internal data interchange format, a registration database incorporating: plural asset data sets (ADS), each including: a registered asset owner and/or its designated third party (RAO) identifier, a geographic location identifier, and carbon content specifications required to record physical characteristics and quantity of sequestered carbon; and also incorporating RAO access credentials for accessing the server;
specifying Gaussian and/or logical transformation models in object code stored as data in one of its non-transitory storage media, the models representing normalization procedures for input data; logical checks for legal compliance; and allocations for accounting purposes; which models will be interpreted by executable code at runtime;
generating a new ADS in the registration database, in response to a user request to become an RAO, the user request communicated to a portal of the server, via an application programming interface (API) or enterprise resource planning ERP software operating on a communication device of the user, the server communicating RAO access credentials to the user and now recognizing the user as an RAO;
operating a continuous monitoring engine, created and stored in one of its non-transitory storage media, in communication with the registration database, which for each ADS:
upon initial registration of a new ADS, defines initial baseline data needed to quantify the physical characteristics and quantity of sequestered carbon at the registered location;
communicates a request for the defined initial baseline data to and receives them from the RAO or its designated third parties, via its user communication device, and conforms the received initial baseline data to its internal data interchange format, and stores same in one of its non-transitory storage media;
periodically communicates with the RAO or its designated third parties, via its user communication device, to receive and collect new data for an existing ADS, including any changes in any one or more of: quantity of sequestered carbon attributable to physical processes of additional sequestration from and/or release of either previously sequestered or new carbon sources into the environment, and/or transformation of physical properties, and/or transport into or away from the registered sequestration location and/or ownership thereof, conforms the newly received data to its internal data interchange format, and updates the ADS in one of its non-transitory storage media
communicates with the RAO or its designated third parties, via its user communication device, requesting new data to update the existing ADS to conform with modification of ADS specifications or requirements used in operation of the continuous monitoring engine, receives the new data from the RAO, conforms the newly received data to its internal data interchange format, updates the ADS, and stores same in one of its non-transitory storage media;
when required for the ADS, assigns one or more stock keeping units (SKU) to sub-units of the sequestered carbon to monitor material transformation and/or transport thereof in stream of commerce and communicates the SKU data to the RAO, via its user communication device, and stores same in one of its non-transitory storage media;
generates carbon sequestration asset units (SCAUs), linked to each ADS, each SCAU including quantity, quality, characteristics, origination location and current location of sequestered carbon and stores same in one of its non-transitory storage media;
communicates with external data services, including both sensors and external computer accounting systems, associated with specific ADS in the registration database, to collect component input data in native format, and stores same in one of its non-transitory storage media;
transforms data throughout the continuous monitoring process using a model-based interpreter that uses one or more structured transformation models (STMs) specified in object code representing both Gaussian and logical transforms, and stores same in one of its non-transitory storage media;
stores STMs as self-referencing data objects that expose input-output metadata such that the model-based interpreter can automatically load the relevant STMs for a given set of data for a combination of ADS and SCAU such that the interpreter will read the stored data objects and perform Gaussian and logical transforms at runtime, and stores same in one of its non-transitory storage media;
specifies STMs that perform Gaussian and logical transformations on the input data to support physical property calculations, legal compliance tests, and accounting allocations, and stores same in one of its non-transitory storage media;
transforms ADS component data to normalized elements for aggregation and analysis using a model-based interpreter that specifies normalizations specific to data input source and data elements, and stores same in one of its non-transitory storage media;
aggregates and analyzes plural ADS, SKU and SCAU data to derive any temporal changes in ownership, quantity and/or physical properties of sequestered carbon attributable to: additional sequestration from, and/or release of previously sequestered carbon, and/or introduction of new carbon sources into the environment, transformation of physical properties of the sequestered carbon, and/or transport of sequestered carbon into or away from the registered sequestration location using applied STMs in the model-based interpreter, storing the aggregation and analysis data in in one of its non-transitory storage media;
updates each ADS, SKU and SCAU data in a continuous feedback loop and stores same in one of its non-transitory storage media; and
creating a digital ledger, in one of its non-transitory storage media, which is in communication with the continuous monitoring engine, storing in the digital ledger the SCAU data in codified data structures that are in compliance with governmental requirements for the generation of tradeable carbon credits, and updating, in the digital ledger in a continuous feedback loop in conjunction with the continuous monitoring engine, the SCAU data, and the codified data structures for compliance with changes in the governmental requirements.

17. The method of claim 16, further comprising the digital ledger communicating with a carbon continuous settlement engine for facilitating trading of sequestered carbon credits associated with the SCAU data in the digital ledger, and the digital ledger modifying SCAU data to reflect changes in ownership of and/or offsetting consumption of sequestered carbon.

18. The method of claim 17, further comprising the continuous monitoring engine tagging an SCAU, its related data set, and its related ADS with a compliance code that it complies with one or more governmental requirements for the generation of tradeable carbon credits.

19. The method of claim 17, further comprising the continuous monitoring engine executing application of runtime-interpreted, object specific Gaussian and logical models for logical assertion of compliance with a given authority.

20. A computer program product, comprising a first non-transitory, computer-readable storage medium. including instructions which, when executed by one or more processors in a server coupled to the first medium and to a second non-transitory storage media, cause the server to execute the method of claim 16 for monitoring and generating asset data sets (ADS) concerning properties and characteristics of sequestered carbon, and archiving same in a database.
